(19) United States
(12) Patent Application Publication
(10) Pub. No.: US 2026/0263134 A1
(43) Pub. Date: Sep. 10, 2026

(54) BALLOON CATHETER COMPONENT LOCATING MARKERBAND

(71) Applicant: Medtronic Ireland Manufacturing Unlimited Company, Dublin (IE)

(72) Inventors: Carlos H. Lima, Santa Rosa, CA (US); Gergo Edes, Fort Worth, TX (US)

(21) Appl. No.: 19/472,533

(22) PCT Filed: Mar. 25, 2024
(86) PCT No.: PCT/EP2024/057904

§ 371 Date: Oct. 3, 2025

Related U.S. Application Data
(60) Provisional application No. 63/494,383, filed on Apr. 5, 2023.

(51) Int. Cl. A61B 18/02; A61B 17/00; A61B 18/00; A61B 90/00

(52) U.S. Cl. CPC A61B 18/02; A61B 90/39; A61B 2017/00526; A61B 2017/00557; A61B 2018/00172; A61B 2018/00577; A61B 2018/00821; A61B 2018/0212; A61B 2090/3966

(57) ABSTRACT

A catheter including an inner member, an auxiliary member, and a markerband. The markerband includes a main body defining a central opening and the inner member extends through the central opening such that the main body of the markerband extends around a portion of the inner member. The markerband also includes a first support protrusion extending away from the main body of the markerband and a second support protrusion extending away from the main body of the markerband and spaced apart from the first support protrusion by a gap. The auxiliary member is positioned within the gap between the first support protrusion and the second support protrusion.

### FIELD

The present technology is generally related to cryoablation catheters.

### BACKGROUND

Renal denervation and similar balloon cryotherapies are performed by inflating a balloon with a refrigerant to remove heat from surrounding tissues. The balloon is supported by a catheter which provides the refrigerant to the balloon to perform the therapy. Introduction of the refrigerant to the balloon causes the balloon to expand, and the temperature within the balloon is monitored throughout the therapy process. The catheter includes a radiopaque markerband for locating components of the catheter during the therapy process.

### SUMMARY

The techniques of this disclosure generally relate to the use of a markerband within a balloon of a balloon catheter. The markerband positions an auxiliary member (e.g., an inflow pipe, a thermocouple wire, a pressure tube, or any other suitable catheter component) relative to an inner member of the catheter, and helps secure the auxiliary member relative to the inner member. The markerband stabilizes the auxiliary member on the exterior of the inner member and eases the assembly of the balloon portion of the balloon catheter. The markerband may be made at least partially of a radiopaque material (e.g., gold, platinum, iridium, or other sufficient radiopaque materials).

In one aspect, the disclosure provides a catheter including an inner member, an auxiliary member, and a markerband. The markerband includes a main body defining a central opening. The inner member extends through the central opening such that the main body of the markerband extends around a portion of the inner member. A first support protrusion extends away from the main body of the markerband and a second support protrusion extends away from the main body of the markerband and is spaced apart from the first support protrusion by a gap. The auxiliary member is positioned within the gap between the first support protrusion and the second support protrusion.

In another aspect, the disclosure provides a markerband for a catheter. The markerband including a main body including a first axial end face and a second axial end face. The second axial end face is disposed opposite the first axial end face. The markerband also includes a central opening extending through the main body between the first axial end face and the second axial end face. The central opening is sized and shaped and configured to receive an inner member of the catheter. The markerband also includes a first support protrusion that extends between the first axial end face and the second axial end face. The first support protrusion extends from the main body of the markerband. The markerband also includes a second support protrusion that extends between the first axial end face and the second axial end face. The second support protrusion extends from the main body of the markerband and is spaced apart from the first support protrusion by a gap. The first support protrusion and the second support protrusion are sized and shaped and configured receive an auxiliary member of the catheter within the gap.

In another aspect, the disclosure provides a method of manufacturing a catheter. The method includes inserting an inner member of the catheter through a central opening of a main body of a markerband and inserting an auxiliary member of the catheter between a first support protrusion and a second support protrusion of the markerband. The first support protrusion extends from the main body and the second support protrusion extends from the main body. The method also includes securing the auxiliary member in place relative to the first support protrusion and the second support protrusion.

Further disclosed herein is a catheter including an inner member, an auxiliary member, and a markerband, wherein the markerband includes a main body defining a central opening and the inner member extends through the central opening such that the main body of the markerband extends around a portion of the inner member, wherein the markerband also includes a first support protrusion extending away from the main body of the markerband and a second support protrusion extending away from the main body of the markerband and spaced apart from the first support protrusion by a gap, and wherein the auxiliary member is positioned within the gap between the first support protrusion and the second support protrusion.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a perspective view of a distal end of a cryoablation catheter according to one example.

FIG. 2 is a close-up perspective view of the distal end of the cryoablation catheter of FIG. 1, including a markerband in accordance with one example.

FIG. 3 is a front view of the markerband of FIG. 2 according to one example.

FIG. 4 is a front view of the markerband of FIG. 2 in accordance with another example.

FIG. 5 is a front view of a markerband in accordance with another example.

FIG. 6 is a flow chart of a method of manufacturing a catheter including a markerband, according to one example.

### DETAILED DESCRIPTION

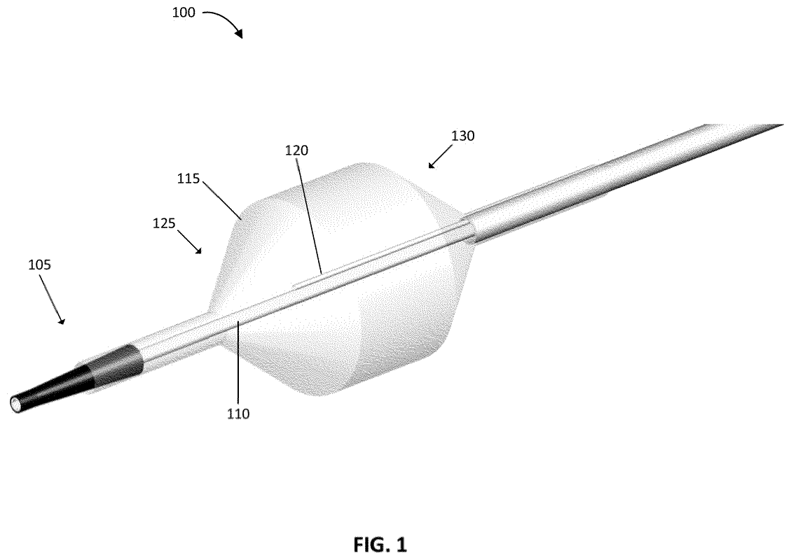

With reference to FIGS. 1-4, a medical device, illustrated as a catheter 100 (e.g., a cryoablation catheter, a renal denervation catheter, or any suitable balloon catheter) includes a distal end 105 sized and shaped to be inserted into the vasculature of a patient and a proximal end (not shown) to be engaged and controlled by an operator. The distal end 105 includes an inner member 110 surrounded, in part, by a balloon 115 that is selectively inflatable (e.g., to perform cryoablation therapy). In some examples, the catheter 100 delivers a refrigerant to the balloon 115 to inflate and cool the balloon 115 during a cryoablation therapy. An auxiliary member 120 is disposed within the catheter 100 and extends to a position within the balloon 115, for example between a distal end 125 of the balloon 115 and a proximal end 130 of the balloon 115. The auxiliary member 120 may for example be a component of the catheter 100 that is typically disposed within the inner member 110, but is disposed outside of the inner member 110 (as shown in FIG. 1) to allow for a smaller diameter of the inner member 110. In some examples, the auxiliary member 120 is an inflow pipe for introducing the refrigerant to the balloon 115. In such examples, the auxiliary member 120 may include a plurality of exhaust ports that deliver the refrigerant to a volume of space within the balloon 115 during use. The exhaust ports are openings (e.g., radial openings) in the auxiliary member 120 and direct the refrigerant radially outward toward the balloon 115. The refrigerant flows through the auxiliary member 120 of the catheter 100 as a liquid and undergoes a liquid-gas phase change when passing through the exhaust ports and entering the balloon 115. The phase change results in rapid expansion of the now gaseous refrigerant, thereby decreasing the temperature within the balloon 115 and inflating the balloon 115 (e.g., near the distal end 105).

Although illustrated as an inflow pipe, it should be understood that the auxiliary member 120 may be a different component of the catheter 100. For example, the auxiliary member 120 may be a thermocouple wire to allow for monitoring of the internal temperature of the balloon 115; a pressure tube, including a pressure sensor, to allow for monitoring of the internal pressure of the balloon 115; an exhaust tube to allow exhaust of fluid from the balloon 115; etc.

The catheter 100 also includes a markerband 140 that is disposed within the balloon 115. The markerband 140 is formed of a radiopaque material such as, but not limited to, gold, platinum, iridium, or other radiopaque materials to allow the distal end 105 (or components thereof) of the catheter 100 to be tracked using fluoroscopy (e.g., while in use for a cryoablation therapy). Although illustrated as a single markerband 140, it should be understood that, in some examples, the catheter 100 includes a greater number of markerbands 140 than illustrated. The markerband 140 is positioned within the balloon 115 and is secured to the inner member 110. With reference to FIG. 1, the markerband 140 may be located near the distal end 125 of the balloon 115, the proximal end 130 of the balloon 115, or for example extend the length of the balloon 115. Although described herein as positioned within the balloon 115, the catheter 100 may include one or more markerbands 140 along the length of the catheter 100 at locations other than within the balloon 115. In some instances, the auxiliary member 120 is secured (e.g., with or without adhesive) to the markerband 140 to locate (e.g., to prevent movement) of the auxiliary member 120 relative to the inner member 110.

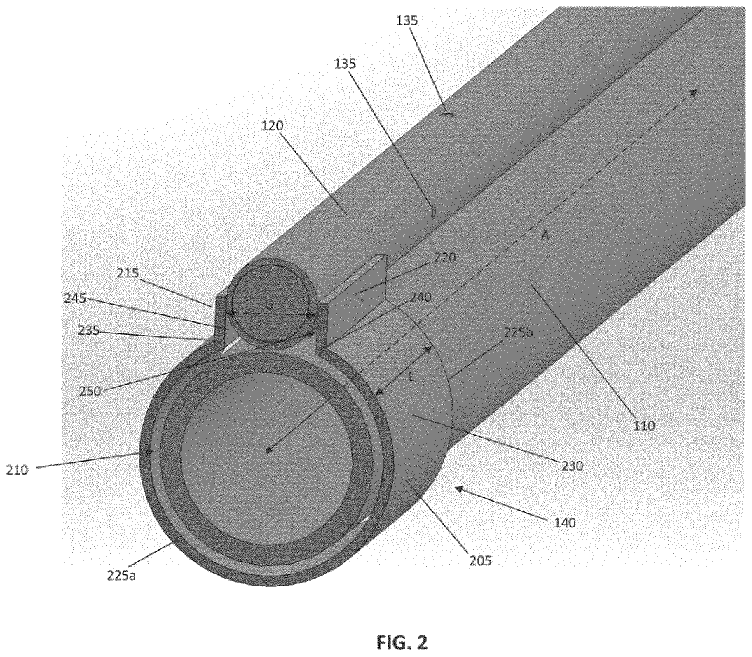

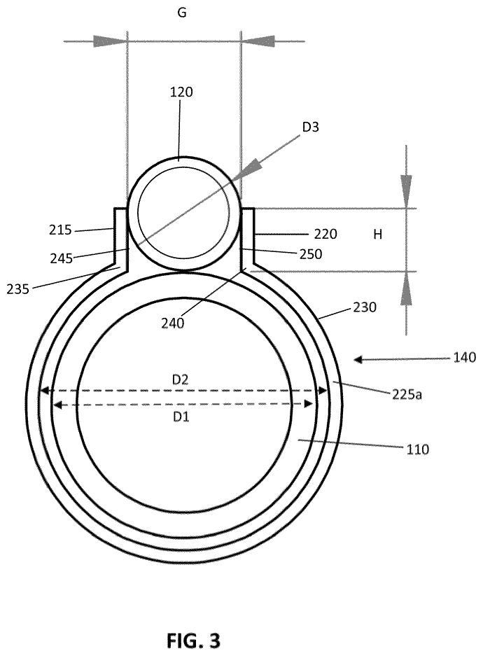

With reference to FIGS. 2-3, in some examples the markerband 140 is sized and shaped to support the auxiliary member 120 relative to the inner member 110. In the illustrated example, the markerband 140 includes a main body 205 defining a central opening 210. The central opening 210 is sized and shaped such that the inner member 110 may extend through the central opening 210. For example, in the illustrated example, the main body 205 extends (e.g., substantially extends) around a portion of the inner member 110. The markerband 140 also includes a first support protrusion 215 (e.g., a first arm) and a second support protrusion 220 (e.g., a second arm). The first support protrusion 215 extends away from the main body 205 and away from the central opening 210 in a first direction. The second support protrusion 220 extends away from the main body 205 and the central opening 210 in a second direction. In some examples, and as illustrated in FIG. 2, the first direction is parallel to the second direction. The second support protrusion 220 is spaced apart from the first support protrusion 215, along the main body 205, by a gap G. The gap G is a distance between the first support protrusion 215 and the second support protrusion 220. As shown in FIGS. 2-3, the auxiliary member 120 is positioned (e.g., secured) within the gap G between the first support protrusion 215 and the second support protrusion 220. The auxiliary member 120 and the marker band 140 (including the main body 205 and the first and second support protrusions 215, 220) may be sized and shaped such that the auxiliary member 120 engages both the first support protrusion 215 and the second support protrusion 220. In some instances, without the first support protrusion 215 and the second support protrusion 220, the auxiliary member 120 may move relative to the surface of the inner member 110. By positioning the auxiliary member 120 within the gap G, the first support protrusion 215 and the second support protrusion 220 facilitate positioning of the auxiliary member 120 relative to the inner member 110 and the markerband 140 and allow the auxiliary member 120 to be secured relative to the inner member 110.

With continued reference to FIGS. 2-3, the main body 205 includes a first axial end face 225a and a second axial end face 225b. The second axial end face 225b is disposed opposite the first axial end face 225a on the main body 205. The second axial end face 225b is spaced apart from the first axial end face 225a by a length L of the main body 205. In some examples, the length L is the length of the markerband 140 from the first axial end face 225a to the second axial end face 225b. In the illustrated example, the central opening 210 extends between the first axial end face 225a and the second axial end face 225b. As illustrated in FIG. 2, the main body 205 extends along a longitudinal axis A of the catheter 100. As such, the central opening 210 extends between the first axial end face 225a and the second axial end face 225b along the longitudinal axis A. Likewise, both the first support protrusion 215 and the second support protrusion 220 extend between the first axial end face 225a and the second axial end face 225b along the longitudinal axis A. The main body 205 also includes an outer profile 230. When the main body 205 is viewed along the longitudinal axis A, as shown in FIG. 3, the outer profile 230 is an arc of a circle in shape, as defined by the gap G. In some examples, the main body 205 includes a first circumferential end 235 and a second circumferential end 240. The first circumferential end 235 and the second circumferential end 240 terminate the arcuate shape of the outer profile 230. As described above, the first support protrusion 215 and the second support protrusion 220 extend away from the main body 205. In some examples, the first support protrusion 215 extends from the first circumferential end 235 away from the main body 205. Likewise, in some examples, the second support protrusion 220 extends from the second circumferential end 240 away from the main body 205.

With continued reference to FIG. 3, the inner member 110 has a first, outer diameter D1. The central opening 210 of the main body 205 has a second, inner diameter D2. In some examples, the first, outer diameter D1 is less than or equal to the second, inner diameter D2 to allow for the inner member 110 to extend through the central opening 210, as described above, without interference from the main body 205. In other examples, the diameter D2 is less than the diameter D1, such that the markerband 140 is crimped over the inner member 110 and is held on the inner member 110 for example via a friction fit. The auxiliary member 120 has a third, outer diameter D3. In some examples, the third, outer diameter D3 is equal to or less than the gap G to allow for the auxiliary member 120 to be positioned between the first support protrusion 215 and the second support protrusion 220 without interference from the first support protrusion 215 and the second support protrusion 220. In other examples, the diameter D3 is greater than the gap G, such that the first and second support protrusions 215, 220 are flexed apart from one another when the auxiliary member 120 is inserted into the gap G. As shown in FIG. 3, the first support protrusion 215 has a first height H. Likewise, in the illustrated example, the second support protrusion 220 has a second height that is equal to the first height H. In some examples, the first height His at least half of the third, outer diameter D3 of the auxiliary member 120. The first height H stabilizes the auxiliary member 120 between the first support protrusion 215 and the second support protrusion 220 to inhibit or prevent movement of the auxiliary member 120 relative to the inner member 110. In some examples, the height of the first support protrusion 215 is different than the height of the second support protrusion 220.

Referring back to FIG. 2, the first support protrusion 215 includes a first inner wall 245. The first inner wall 245 is positioned opposite the outer profile 230 on the first support protrusion 215, and is positioned to face the gap G. In some examples, the first inner wall 245 is a portion of the first support protrusion 215 in physical contact with the auxiliary member 120 when the auxiliary member 120 is positioned within the gap G. Likewise, the second support protrusion 220 includes a second inner wall 250. The second inner wall 250 is positioned opposite the outer profile 230 on the second support protrusion 220, and is positioned to face the gap G and the first inner wall 245. In some examples, the second inner wall 250 is a portion of the second support protrusion 220 in physical contact with the auxiliary member 120 when the auxiliary member 120 is positioned within the gap G. When the auxiliary member 120 is positioned within the gap G, the auxiliary member 120 may be secured to the first inner wall 245 and the second inner wall 250 to further prevent movement of the auxiliary member 120 relative to the inner member 110. In some examples, the auxiliary member 120 is secured to the first inner wall 245 and the second inner wall 250 with an adhesive. The adhesive may be an epoxy-based adhesive, acrylic-based adhesive, a silicone-based adhesive, a polyurethane-based adhesive, or any other suitable medical grade adhesive. As shown in FIGS. 2-3, the auxiliary member 120 is in physical contact with the inner member 110. When the auxiliary member 120 is positioned within the gap G, the auxiliary member 120 may be secured to the inner member 110. For example, the auxiliary member 120 may be secured to the inner member 110 with the adhesive. In other examples, the auxiliary member 120 remains spaced from the inner member 110, and/or is not secured to the inner member 110.

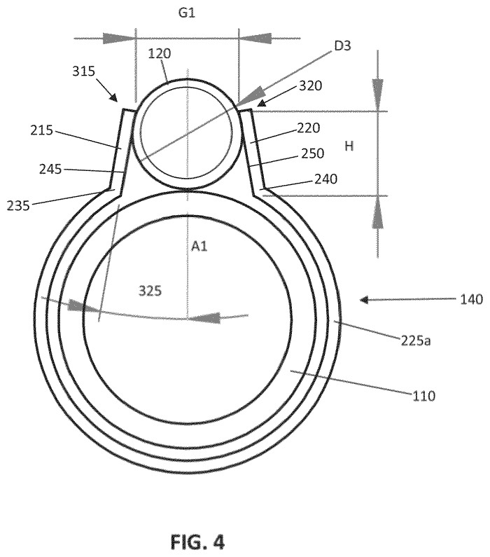

With reference to FIG. 4, in some examples, the first support protrusion 215 and the second support protrusion 220 are non-parallel. In the illustrated example, the first support protrusion 215 includes a first distal end 315. The second support protrusion 220 includes a second distal end 320. The first distal end 315 is spaced from the main body 205 by the first height H of the first support protrusion 215. Likewise, the second distal end 320 is spaced from the main body 205 by the second height of the second support protrusion 220. Similar to the example shown in FIG. 3, the second height H is equal to the first height H and is at least half the third, outer diameter D3 of the auxiliary member 120. In the illustrated example of FIG. 4, the first support protrusion 215 extends at a first angle 325 relative to an axis A1 that is both perpendicular to the longitudinal axis A and equidistant from both the first distal end 315 and the second distal end 320. In some examples, the first angle 325 is less than 90 degrees. Likewise, the second support protrusion 220 extends at a second angle relative to the axis A1. The second angle is equal to and opposite in magnitude to the first angle 325. Accordingly, the first support protrusion 215 and the second support protrusion 220 are angled toward one another at the first distal end 315 and the second distal end 320. In the illustrated example, the first distal end 315 is spaced apart from the second distal end 320 by a gap G1. As shown in FIG. 4, the gap G1 between the first distal end 315 and the second distal end 320 is less than the third, outer diameter D3 of the auxiliary member 120, such that the auxiliary member 120 is secured between the first inner wall 245 and the second inner wall 250. In some examples, the auxiliary member 120 is kept in place by frictional engagement between the auxiliary member 120 and the first inner wall 245 and second inner wall 250, respectively. In other examples, the auxiliary member 120 is secured to the first inner wall 245 and the second inner wall 250 with adhesive.

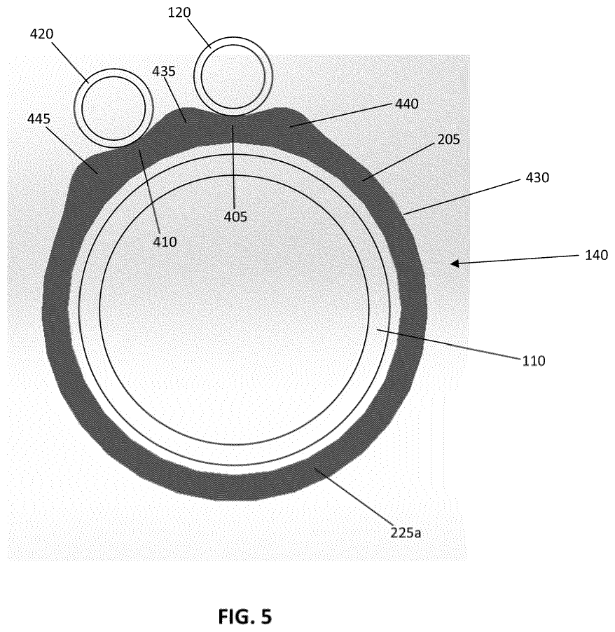

With reference to FIG. 5, in some examples, the main body 205 has a continuous outer profile 430. In the illustrated example, the continuous outer profile 430 is shaped to completely surround the inner member 110. In other words, the continuous outer profile 430 extends entirely around (e.g., circles) an exterior of the main body 205. In the illustrated example, the main body 205 (or at least a portion thereof) is generally circular in shape, although in other examples the main body 205 has an elliptical shape or other shape. As shown in FIG. 5, in the illustrated example, the continuous outer profile 430 includes a first support protrusion 435 and a second support protrusion 440. In some examples, such as the illustrated example, the first support protrusion 435 and the second support protrusion 440 may be rounded in shape. In some examples, the first support protrusion 435 and the second support protrusion 440 are shaped differently than the first support protrusion 215 and the second support protrusion 220, as described above. The continuous outer profile 430 also includes a first groove 405 that extends between the first support protrusion 435 and the second support protrusion 440. The first groove 405 may be integrally formed as part of the main body 205, or for example may be machined down during assembly of the catheter 100 to fit a circumference of the auxiliary member 120. In the illustrated example, the first groove 405 extends between the first support protrusion 435 and the second support protrusion 440 along the continuous outer profile 430. In some examples, the depth of the first groove 405 is less than half of the third, outer diameter D3 of the auxiliary member 120. The auxiliary member 120 is positioned within the first groove 405. In some examples, the auxiliary member 120 is secured to the first groove 405 with adhesive. Positioning the auxiliary member 120 within the first groove 405 allows the auxiliary member 120 to be kept in place between the first support protrusion 435 and the second support protrusion 440 relative to the inner member 110.

With continued reference to FIG. 5, and in some examples, the continuous outer profile 430 includes a second groove 410 and a third support protrusion 445 spaced apart from the first support protrusion 435. The second groove 410 extends between the first support protrusion 435 and the third support protrusion 445, along the continuous outer profile 430. In some examples, the depth of the second groove 410 is the same as the depth of the first groove 405. In some examples, a separate auxiliary member 420 is positioned within the second groove 410. The separate auxiliary member 420 may be an inflow pipe, a thermocouple wire, a pressure tube, or any other suitable catheter component. In some examples, the separate auxiliary member 420 is secured to the second groove 410 with adhesive. Positioning the separate auxiliary member 420 within the second groove 410 allows the separate auxiliary member 420 to be kept in place between the third support protrusion 445 and the first support protrusion 435 relative to the inner member 110. In some examples, the separate auxiliary member 420 is different than the auxiliary member 120. In some examples, the auxiliary member 120 is positioned within the second groove 410 instead of the separate auxiliary member 420.

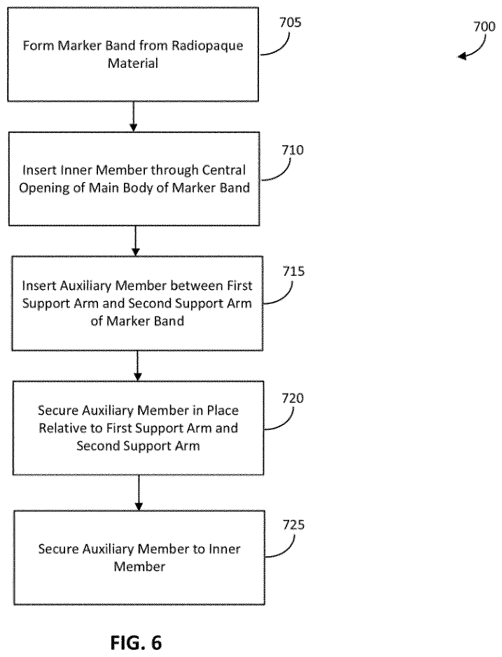

FIG. 6 is a flow chart of a method 700 of manufacturing the catheter 100 including the markerband 140, according to one example. While a particular order is provided, in some examples, the steps of the method 700 may be performed in a different order. Additionally, the method 700 may include a greater or fewer number of steps than provided. The method 700 begins at step 705 when the markerband 140 is formed from a radiopaque material. The method 700 then proceeds to step 710. At step 710, the inner member 110 is inserted through the central opening 210 of the main body 205. In some examples, the inner member 110 is secured to the main body 205 of the markerband 140, or vice versa (e.g., with adhesive). In other examples, the main body 205 is crimped around the inner member 110 to maximize contact between the main body 205 and the inner member 110. The method 700 then proceeds to step 715. At step 715, the auxiliary member 120, 420 is inserted between the first support protrusion 215, 435 and the second support protrusion 220, 440. For example, the auxiliary member 120, 420 is positioned within the gap G, G1. In other examples, the auxiliary member 120, 420 is positioned within the first groove 405. The method 700 then proceeds to step 720.

At step 720, the auxiliary member 120, 420 is secured in place relative to the first support protrusion 215, 435 and the second support protrusion 220, 440. For example, the auxiliary member 120, 420 is secured to the first support protrusion 215, 435 and the second support protrusion 220, 440 with adhesive. In other examples, the auxiliary member 120, 420 is secured to the first support protrusion 215, 435 and the second support protrusion 220, 440 by frictional engagement. Additionally, the first inner wall 245 and the second inner wall 250 may be roughened to allow for greater friction between the auxiliary member 120, 420 and the first support protrusion 215, 435 and the second support protrusion 220, 440. In even other examples, the auxiliary member 120, 420 is secured to the first groove 405 with adhesive. The method 700 then proceeds to step 725. At step 725, the auxiliary member 120, 420 is secured to the inner member 110. For example, the auxiliary member 120 is secured to the inner member 110 with adhesive.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

The invention may be further described by reference to the following numbered paragraphs:
1. A catheter comprising:
an inner member;
an auxiliary member; and
a markerband, the markerband including
   a main body defining a central opening, wherein the inner member extends through the central opening such that the main body of the markerband extends around a portion of the inner member;
   a first support protrusion extending away from the main body of the markerband; and
   a second support protrusion extending away from the main body of the markerband and spaced apart from the first support protrusion by a gap, wherein the auxiliary member is positioned within the gap between the first support protrusion and the second support protrusion.
2. The catheter of paragraph 1, wherein the main body includes a first axial end face and a second axial end face, the second axial end face disposed opposite the first axial end face, wherein the central opening of the main body extends between the first axial end face and the second axial end face, wherein the first support protrusion extends between the first axial end face and the second axial end face, and wherein the second support protrusion extends between the first axial end face and the second axial end face.
3. The catheter of paragraph 1, wherein the inner member has a first, outer diameter and the central opening of the main body has a second, inner diameter, wherein the first, outer diameter is equal to or greater than the second, inner diameter.
4. The catheter of paragraph 1, wherein the auxiliary member has a third, outer diameter, wherein the third, outer diameter is equal to or less than the gap between the first support protrusion and the second support protrusion, and wherein the auxiliary member comprises at least one of an inflow pipe, a thermocouple wire, or a pressure tube.
5. The catheter of paragraph 4, wherein the main body extends along a longitudinal axis and has an outer profile, as viewed along the longitudinal axis, that is an arc of a circle, wherein the main body includes a first circumferential end, and a second circumferential end, wherein the first support protrusion extends from the first circumferential end and the second support protrusion extends from the second circumferential end, wherein the first support protrusion has a first height that is at least half the third, outer diameter of the auxiliary member, and wherein the second support protrusion has a second height that is substantially equal to the first height.
6. The catheter of paragraph 5, wherein the first support protrusion has a first inner wall and the second support protrusion has a second inner wall that faces the first inner wall, and wherein the auxiliary member is secured to the first inner wall and the second inner wall with an adhesive.
7. The catheter of paragraph 6, wherein the auxiliary member is in physical contact with the inner member.
8. The catheter of paragraph 6, wherein the first support protrusion and the second support protrusion are non-parallel.
9. The catheter of paragraph 8, wherein the first support protrusion includes a first distal end spaced from the main body and the second support protrusion includes a second distal end spaced from the main body, wherein the first support protrusion and the second support protrusion are angled toward one another at the first and second distal ends such that a distance between the first distal end and the second distal end is less than the third, outer diameter.
10. The catheter of paragraph 4, wherein the main body has a continuous outer profile that extends entirely around an exterior of the main body, wherein the outer profile includes a groove defined between the first support protrusion and the second support protrusion, and wherein the auxiliary member is positioned within the groove between the first support protrusion and the second support protrusion.
11. The catheter of paragraph 1, wherein the markerband comprises a radiopaque material, and further comprising an inflatable balloon surrounding the inner member.
12. A markerband for a catheter, the markerband comprising:
a main body including a first axial end face and a second axial end face, the second axial end face disposed opposite the first axial end face;
a central opening extending through the main body between the first axial end face and the second axial end face, the central opening sized and shaped and configured to receive an inner member of the catheter;
a first support protrusion extending between the first axial end face and the second axial end face, the first support protrusion extending from the main body of the markerband; and
a second support protrusion extending between the first axial end face and the second axial end face, the second support protrusion extending from the main body of the markerband and spaced apart from the first support protrusion by a gap, wherein the first support protrusion and the second support protrusion are sized and shaped and configured to receive an auxiliary member of the catheter within the gap.
13. The markerband of paragraph 12, wherein the inner member has a first, outer diameter and the central opening has a second, inner diameter, wherein the first, outer diameter is equal to or greater than the second, inner diameter.
14. The markerband of paragraph 12 or of any of paragraphs 12-13, wherein the auxiliary member has a third, outer diameter, wherein the third, outer diameter is equal to or greater than the gap between the first support protrusion and the second support protrusion, and wherein the auxiliary member comprises at least one of an inflow pipe, a thermocouple wire, or a pressure tube.
15. The markerband of paragraph 12 or of any of paragraphs 12-14, wherein the markerband comprises a radiopaque material.
16. A method of manufacturing a catheter comprising:
inserting an inner member of the catheter through a central opening of a main body of a markerband;
inserting an auxiliary member of the catheter between a first support protrusion and a second support protrusion of the markerband, wherein the first support protrusion extends from the main body and the second support protrusion extends from the main body; and
securing the auxiliary member in place relative to the first support protrusion and the second support protrusion.
17. The method of paragraph 16, wherein the auxiliary member is secured to the first support protrusion and the second support protrusion with an adhesive.
18. The method of paragraph 16, further comprising securing the auxiliary member to the inner member.
19. The method of paragraph 16, wherein the auxiliary member is secured to the markerband within a groove between the first support protrusion and the second support protrusion.
20. The method of paragraph 16, further comprising forming the markerband from a radiopaque material.

Although various aspects and examples have been described in detail with reference to certain examples illustrated in the drawings, variations and modifications exist within the scope and spirit of one or more independent aspects described and illustrated.

Further disclosed herein is the subject-matter of the following clauses:
1. A catheter comprising:
an inner member;
an auxiliary member; and
a markerband, the markerband including
   a main body defining a central opening, wherein the inner member extends through the central opening such that the main body of the markerband extends around a portion of the inner member;
   a first support protrusion extending away from the main body of the markerband; and
   a second support protrusion extending away from the main body of the markerband and spaced apart from the first support protrusion by a gap, wherein the auxiliary member is positioned within the gap between the first support protrusion and the second support protrusion.
2. The catheter of clause 1, wherein the main body includes a first axial end face and a second axial end face, the second axial end face disposed opposite the first axial end face, wherein the central opening of the main body extends between the first axial end face and the second axial end face, wherein the first support protrusion extends between the first axial end face and the second axial end face, and wherein the second support protrusion extends between the first axial end face and the second axial end face.
3. The catheter of any of the preceding clauses, wherein the inner member has a first, outer diameter and the central opening of the main body has a second, inner diameter, wherein the first, outer diameter is equal to or greater than the second, inner diameter.
4. The catheter of any of the preceding clauses, wherein the auxiliary member has a third, outer diameter, wherein the third, outer diameter is equal to or less than the gap between the first support protrusion and the second support protrusion, and wherein the auxiliary member comprises at least one of an inflow pipe, a thermocouple wire, or a pressure tube.
5. The catheter of clause 4, wherein the main body extends along a longitudinal axis and has an outer profile, as viewed along the longitudinal axis, that is an arc of a circle, wherein the main body includes a first circumferential end, and a second circumferential end, wherein the first support protrusion extends from the first circumferential end and the second support protrusion extends from the second circumferential end, wherein the first support protrusion has a first height that is at least half the third, outer diameter of the auxiliary member, and wherein the second support protrusion has a second height that is substantially equal to the first height.
6. The catheter of any of the preceding clauses, wherein the first support protrusion has a first inner wall and the second support protrusion has a second inner wall that faces the first inner wall, and wherein the auxiliary member is secured to the first inner wall and the second inner wall with an adhesive.
7. The catheter of any of the preceding clauses, wherein the auxiliary member is in physical contact with the inner member.
8. The catheter of any of the preceding clauses, wherein the first support protrusion and the second support protrusion are non-parallel.
9. The catheter of clause 8, wherein the first support protrusion includes a first distal end spaced from the main body and the second support protrusion includes a second distal end spaced from the main body, wherein the first support protrusion and the second support protrusion are angled toward one another at the first and second distal ends such that a distance between the first distal end and the second distal end is less than a third, outer diameter of the auxiliary member.
10. The catheter of any of the preceding clauses, wherein the main body has a continuous outer profile that extends entirely around an exterior of the main body, wherein the outer profile of the main body includes a groove defined between the first support protrusion and the second support protrusion, and wherein the auxiliary member is positioned within the groove between the first support protrusion and the second support protrusion.
11. The catheter of any of the preceding clauses, wherein the markerband comprises a radiopaque material, and further comprising an inflatable balloon surrounding the inner member.
12. A method of manufacturing a catheter comprising:
inserting an inner member of the catheter through a central opening of a main body of a markerband;
inserting an auxiliary member of the catheter between a first support protrusion and a second support protrusion of the markerband, wherein the first support protrusion extends from the main body and the second support protrusion extends from the main body; and
securing the auxiliary member in place relative to the first support protrusion and the second support protrusion.
13. The method of clause 12, wherein the auxiliary member is secured to the first support protrusion and the second support protrusion with an adhesive.
14. The method of any of clauses 12 or 13, further comprising securing the auxiliary member to the inner member.
15. The method of any of clauses 12-14, wherein the auxiliary member is secured to the markerband within a groove between the first support protrusion and the second support protrusion.

1. A catheter comprising:
an inner member;
an auxiliary member; and
a markerband, the markerband including
a main body defining a central opening, wherein the inner member extends through the central opening such that the main body of the markerband extends around a portion of the inner member;
a first support protrusion extending away from the main body of the markerband; and
a second support protrusion extending away from the main body of the markerband and spaced apart from the first support protrusion by a gap, wherein the auxiliary member is positioned within the gap between the first support protrusion and the second support protrusion.

2. The catheter of claim 1, wherein the main body includes a first axial end face and a second axial end face, the second axial end face disposed opposite the first axial end face, wherein the central opening of the main body extends between the first axial end face and the second axial end face, wherein the first support protrusion extends between the first axial end face and the second axial end face, and wherein the second support protrusion extends between the first axial end face and the second axial end face.

3. The catheter of claim 1, wherein the inner member has a first, outer diameter and the central opening of the main body has a second, inner diameter, wherein the first, outer diameter is equal to or greater than the second, inner diameter.

4. The catheter of claim 1, wherein the auxiliary member has a third, outer diameter, wherein the third, outer diameter is equal to or less than the gap between the first support protrusion and the second support protrusion, and wherein the auxiliary member comprises at least one of an inflow pipe, a thermocouple wire, or a pressure tube.

5. The catheter of claim 4, wherein the main body extends along a longitudinal axis and has an outer profile, as viewed along the longitudinal axis, that is an arc of a circle, wherein the main body includes a first circumferential end, and a second circumferential end, wherein the first support protrusion extends from the first circumferential end and the second support protrusion extends from the second circumferential end, wherein the first support protrusion has a first height that is at least half the third, outer diameter of the auxiliary member, and wherein the second support protrusion has a second height that is substantially equal to the first height.

6. The catheter of claim 1, wherein the first support protrusion has a first inner wall and the second support protrusion has a second inner wall that faces the first inner wall, and wherein the auxiliary member is secured to the first inner wall and the second inner wall with an adhesive.

7. The catheter of claim 1, wherein the auxiliary member is in physical contact with the inner member.

8. The catheter of claim 1, wherein the first support protrusion and the second support protrusion are non-parallel.

9. The catheter of claim 8, wherein the first support protrusion includes a first distal end spaced from the main body and the second support protrusion includes a second distal end spaced from the main body, wherein the first support protrusion and the second support protrusion are angled toward one another at the first and second distal ends such that a distance between the first distal end and the second distal end is less than a third, outer diameter of the auxiliary member.

10. The catheter of claim 1, wherein the main body has a continuous outer profile that extends entirely around an exterior of the main body, wherein the outer profile of the main body includes a groove defined between the first support protrusion and the second support protrusion, and wherein the auxiliary member is positioned within the groove between the first support protrusion and the second support protrusion.

11. The catheter of claim 1, wherein the markerband comprises a radiopaque material, and further comprising an inflatable balloon surrounding the inner member.

12. A method of manufacturing a catheter comprising:
inserting an inner member of the catheter through a central opening of a main body of a markerband;
inserting an auxiliary member of the catheter between a first support protrusion and a second support protrusion of the markerband, wherein the first support protrusion extends from the main body and the second support protrusion extends from the main body; and
securing the auxiliary member in place relative to the first support protrusion and the second support protrusion.

13. The method of claim 12, wherein the auxiliary member is secured to the first support protrusion and the second support protrusion with an adhesive.

14. The method of claim 12, further comprising securing the auxiliary member to the inner member.

15. The method of claim 12, wherein the auxiliary member is secured to the markerband within a groove between the first support protrusion and the second support protrusion.
